(19) United States
(12) Patent Application Publication
(10) Pub. No.: US 2010/0047788 A1
(43) Pub. Date: Feb. 25, 2010

(54) METHOD AND KIT FOR DIAGNOSIS AND MONITORING OF THE TREATMENT OF CHRONIC TRIPANOSSOMIASIS AND CHAGAS DISEASE

(75) Inventor: Antonio Raimundo Lima Cruz Teixeira, Brasilia (BR)

Correspondence Address: SUGHRUE MION, PLLC, 2100 PENNSYLVANIA AVENUE, N.W., SUITE 800, WASHINGTON, DC, 20037, US

(73) Assignee: FUNDACÃO UNIVERSIDADE DE BRASÍLIA CDTCAMPUS UNIVER, Brasilia (BR)

(21) Appl. No.: 12/438,106

(22) PCT Filed: Aug. 24, 2007
(86) PCT No.: PCT/BR2007/000214

§ 371 Date: Jul. 2, 2009

(30) Foreign Application Priority Data
Aug. 24, 2006 (BR) PI0603872-7

(51) Int. Cl. C12Q 1/68

(52) U.S. Cl. 435/6

(57) ABSTRACT

The present invention is related to the diagnosis and monitoring of the tripanossomiasis treatment aiming to identify the severity of the disease and to make possible the choice of a treatment with the purpose of curing of individuals, suffering from chronic disease, that were infected by the protozoa of the Trypanosomatidae family.

The method of diagnosis and monitoring of the chronic tripanossomiasis treatment, including the chronic Chagas disease, comprises the following stages: (i) to determine the specific markers of the kDNA integration of the Trypanosomatidae family protozoa into the genome of the host; (ii) to extract the genomic DNA of the infected individual sample; (iii) to cleave the genomic DNA with enzymes of specific restrictions and to separate the mixture of resultant fragments; (iv) to submit the fragments under purification and under the analysis of Southern blot; (v) to hybridize the fragments with a complementary probe (kCR) radiomarked and (vi) to carry through the comparative analysis of the fragments profile of the hybridized genomic DNA with the specific markers of the kDNA integration of the Trypanosomatidae family parasite.

The invention also includes a kit for diagnosis and monitoring of a chronic tripanossomiasis, including the chronic Chagas disease.

### FIELD OF THE INVENTION

The present invention is related to the diagnosis and monitoring of the tripanossomiasis treatment aiming to identify the severity of the disease and to make possible the choice of a treatment with the purpose of curing of individuals, suffering from chronic disease, that were infected by the protozoa of the Trypanosomatidae family. More specifically, the invention deals with methods of kDNA integration identification of protozoa minicircles, including the T. cruzi in the host genome through specific markers of the stated integration.

### BACKGROUND OF THE INVENTION

In the Trypanosomatidae family the parasites of high medical and veterinary relevance are enclosed. They are: (i) the Trypanosoma cruzi that produces the Chagas disease in Americas, (II) the Trypanosoma brucei that is the causing agent of the Sleeping disease in Africa and (III) the Leishmania species that are responsible for leishmaniasis in the whole world.

The Chagas disease, caused for the T. cruzi protozoa, affects 18 million people in Latin America, what represents a local social responsibility of six billion dollar (WHO. Control of Chagas Disease. Technical Report Series 811, 1991). The decurrent morbidity and lethality of the Chagas disease are higher than that produced by the malaria, Schistosomiasis and leishmaniasis (Schmunis, Medicine: 59:125-134, 1999). In Brazil, six million people are carriers of the disease.

The T. cruzi is transmitted to the mammals through triatomines and the infection occurs for the invasion of phagocytic cells, which can be transmitted for other individuals through blood transfusion or vertical transmission (from mother to son through the placenta). The pathological injuries found in the heart and digestive system of patients of chronic Chagas disease can be hidden for decades after the initial infection, not being exclusively explained for the destruction of the host tissue injured for the active parasite. The unit of minimum rejection, that consists of host target cells, free of parasite, lysed for mononuclear cells of the imunologic system, has been a common denominator of the Chagas disease pathology.

In the last decade, the Chagas disease treatment was the reason of controversy because of the little knowledge about the chronic disease phatogenesis and the characteristic injuries that were formed in the Chagas disease patients. There was a questioning on the relevance of the antiparasitic treatment in the clinical handling of this disease (Zhang L, Tarleton R L 1999. Parasite persistence correlates with disease severity and localization in chronic Chagas' disease. J Infect Dis 180:480-6). However, the studies on autoimmunity as an important factor in the disease phatogenesis never excluded the persistence of the parasite in the human host (Tarleton R L 2003. Chagas disease: it rolls it will be autoimmunity? Trends Parasitol 19:447-51). Since then, the consensus on the necessity of developing truthly efficient therapeutical projects on the basis of the premise that the elimination of the parasitic load would directly prevent injuries produced by parasite is getting stronger. Moreover, they would help to interrupt the auto-immune reactions that support the serious injuries that frequently are associated with the individual death (Urbina J. A and Docampo, R. 2003. Specific chemotherapy of Changes disease: controversies and advances. Trends in Parasitol. 19:495-500).

The T. cruzi is characterized by the presence of an only one mithocondria with double adhesive tape maxicircles and minicircles topologycally linked that constitute almost 15% of the cellular DNA, representing the biggest amount of extracellular genetic material in any cell (Lukes J, Guilbride D L, Votypka J, Zikova A, Benne R, Englund P T 2002. Kinetoplast DNA network: evolution of an improbable structure. Euk Cell 1: 495-502; Liu B, Liu Y, Motyka S A, Agbo E E, Englund P T 2005. Fellowship of the rings: the replication of kinetoplast DNA. Trends Parasitol 21: 363-9; Junqueira A C, Degrave W, Brandao A 2005. Minicircle organization and diversity in Trypanosoma cruzi populations. Trends Parasitol 21: 270-2). This net of kinetoplast DNAs is composed of some dozens of maxicircles that codify two big ribossomic RNAs and a hydrophobic mitocondrials protein subgroup (Westenberger S J, Cerqueira G, E l-Sayed N M, Zingales B, Campbell D A, Sturm N R 2006. Mitochondrial maxicircles display conservation of gene order and a conserved element in the non-coding region in Trypanosoma cruzi. BMC Genomics in press), together with thousands of minicircles that supply the information for the after-transcription process of the edited RNA in the form of guide RNAs (Avila H A, Simpson L 1995. Organization and complexity of minicircle-encoded guide RNAs in Trypanosoma cruzi. RNA 1: 939-47; Simpson A G B, Gill E E, Callahan H A, Litaker R W, Roger A J 2004. Early evolution within kinetoplastids (Euglenozoa), and the late emergence of trypanosomatids. Protist 155: 407-422). Each minicircle has four conserved regions of 122-pb interdisperses through four variable regions that contain guide RNA genes, totalizing approximately 1400 pb (Sturm N R, Degrave W, Morel C, Simpson L 1989. Sensitive detection and schizodeme classification of Trypanosoma cruzi cells by amplification of kinetoplast minicircle DNA sequences: use in diagnosis of Chagas' disease. Mol Biochem Parasitol 33: 205-14).

The genetic transference of DNA between eucariotes of different kingdoms was already demonstrated (Nitz N, Gomes C, Rosa A C, D'Souza-Ault M R, Moreno F, Lauria-Pires L, Nascimento R J, Teixeira, A R L 2004. Heritable kDNA integration minicircle sequences from Trypanosoma cruzi into the avian genome: Insights into human Chagas disease. Cell 118: 175-86). Particularly, these authors had demonstrated that the T. cruzi intracellular parasite mitocondrial (kDNA) kinetoplast DNA is transferred to human patients with the Chagas disease. The authors also suggest that the kDNA integration represents a potential cause of the developed auto-immune reply in a significant parcel of patients with chronic Chagas disease. It was demonstrated that kinetoplastic T. cruzi protozoa has the capacity of integrating the sequences of kDNA minicircle in the LINE-1 of mammals genomes (Nitz et al., 2004).

The LINEs are considered some amongst the oldest and well-succeeded creations in the genomes of eukaryotes genomes and a resistant evolutionary force for its capacity to create lineage (Smit A F A, Toth G, Riggs A D, Jurka J 1995. Ancestral mammalian wide subfamilies of LINE-1 repetitive sequences. J Mol Biol 246: 401-17); Kazazian Jr H H 2000. L1 retrotransposons shape the mammalian genome. Science 289: 1152-6; Jurka, J. (2000). Rapbase update: a database and an electronic journal of repetitive elements. Trends Genet. 16, 418-420; Cassavant N C, Scott L, Cantrell M A, Wiggins L E, Baker R J, Wichman H A 2000. The end of the LINE?: Lack of recent L1 activity in a group of South American rodents. Genetics, 154: 1809-17). These elements are associated with retrotransposition, which is involved in insertion mutagenesis and, therefore, in the integration and perpetuation of bacteria deriving genes in the human genome throughout the evolution (Anderson, J. O., Doolitle, W. F. e Nesbo, C. L. (2001). Are there bugs in our genome? Science 292, 1848-1850). Também já foi demonstrado que a progênie de macrófagos transfectados com kDNA de T. cruzi retêm inserções de kDNA em seqüências de ORF-2 de LINE tendo elevada identidade com transcriptase reversa It was also demonstrated that the lineage of transfected macrophages with T. cruzi kDNA holds back insertions of kDNA in sequences of ORF-2 of LINE having great identity with reverse transcriptase (Nitz et al., 2004).

This discovery allows deducing that the kDNA inside the LINE-1 was moved through an active precursor of RNA. In fact, it was demonstrated that the AZT prevented the retrotransposition of LINE-1, what is an indication of non occurrence of the kDNA integration (Nitz et al., 2004).

Although all the already done research, great part of the host cell machinery and parasite involved in the transference, the integration and perpetuation of the T. cruzi DNA inside of the host genome is still unknown (International Human Genome Sequencing Consortium 2001. Initial sequencing and analysis of the human genome. Nature 409: 860-927; Venter C G, Adams M D, Myers E W, Li P W, Mural R J, Sutton G G, Smith H O, Yandell M, Evans C A, Holt R A et al. 2001. The sequence of the human genome. Science 291: 1304-51).

The key-points in the human and veterinary medicine are those associated with the sprouting of symptoms and signals that can be recognized as clinical aspects of a disease. Any persistently infectious biological process can be divided into as many severity grade as are the necessary measures to alliviate the symptoms and to treat the signals. In the case of the infections caused by T. cruzi, this process can be divided in two severity grades that succeeds each other, the acute and the chronic.

The marked difference in the antiparasitic activity in the acute and chronic phases of the T. cruzi infection shows the necessity of differentiated treatment modalities development (Braga M S, Lauria-Pires L, Arganaraz E R, Nascimento R J, Teixeira A R. (2000). Persistent infections in chronic Chagas' disease patients treated with anti-Trypanosoma cruzi nitroderivatives. Rev Inst Med Trop Sao Paulo. May-June; 42(3):157-61; Lauria-Pires, L., Braga, M. S., Vexenat, A. C., Simões-Barbosa, A. S., Tinoco, D. L., and Teixeira, A. R. L. (2000). Progressive chronic Chagas heart disease ten years after treatment with anti-Trypanosoma cruzi nitroderivatives. Am. J. Trop. Med. Hyg. 63, 43-55; Urbina & Docampo, 2003). Urbina and friends. (1996) (Urbina, J. A., Payares, G., Molina, J., Sanoja, C., Liendo, A., Lazaardi, K., Piras, M. M., Piras, R., Perez, N., Wincker, P., Ryley, J. F. 1996. Cure of short- and long-term experimental Chagas' disease useing DO870. Science, 273: 969-971).

Summarizing, for an efficient treatment of the infected patient through a Trypanosomatidae family protozoa, including the responsible for the Chagas disease, it is basic to diagnosis in which development phase the disease are, being equally important to make the treatment monitoring, especially in the chronic phase of the disease, to eliminate or to reduce to the minimum the occurrence autoimmunity mechanisms causing deleterious effects, and even though lethal, in the patient.

### SUMMARY OF THE INVENTION

It was now verified that the efficient treatment of infection caused by the Trypanosomatidae family protozoa, including the etiologic agent of the Chagas disease, requires the necessary identification of the occurrence of protozoa minicircles kDNA integration, including the T. cruzi, in the host genome. This is the prove that the disease is already in its chronic phase, requiring, therefore, a regimen of treatment differentiated from that currently known and that has shown itself reasonable efficient in the acute phase, but that does not overcome the disease in the chronic phase. Moreover, it is essential the treatment monitoring by means of a disgnostic method that can detect specific markers of protozoa minicircles kDNA integration, including the T. cruzi, in the host genome.

A first concretization of the present invention is related to the diagnosis method of the Chagas disease in the chronic phase comprising the use of specific genomic markers for the T. cruzi kDNA integration ocorrence determination in the host genome.

In a second concretization of the invention it is provided a Chagas disease treatment monitoring method in the chronic phase comprising the use of specific genomic markers for the T. cruzi kDNA integration ocorrence determination in the host genome.

In a third concretization of the invention an identification method is provided of the chronic phase of disease caused by a Trypanosomatidae family protozoa and of the monitoring treatment of the stated disease comprising the specific markers of the kDNA integration related member of stated family in the host genome.

The fourth concretization of the invention is related to the provisions of a diagnosis kit for the identification of the Chagas disease chronic phase and for the treatment monitoring comprising the specific markers of the T. cruzi kDNA integration in the host genome; reagents for the extraction of DNA and analysis of the resultant fragments of cleavage with specific enzymes of restriction and instructions for the accomplishment of the extraction and analysis of fragments.

A fifth concretization of the invention is related to the provisions of a diagnosis kit for the identification of the chronic phase of disease caused for one of the members of the Trypanosomatidae family and for the monitoring of its treatment comprising the specific markers of the kDNA integration of the related member of the stated family in the host genome; reagents for extraction of DNA and analysis of the resultant fragments of cleavage with specific enzymes of restriction and instructions for the accomplishment of the extraction and analysis of fragments.

### BRIEF DESCRIPTION OF THE DRAWINGS

FIG. 1 shows the integration of the Trypanosoma cruzi minicircles in the genome of macrophages in consequence of the infection.

FIG. 2 illustrates the co-localization of the kDNA minicircle sequences of Trypanosoma cruzi into the macrophage chromosomes after-infection in metaphase plate.

FIG. 3 illustrates the schematical representation of the Trypanosoma cruzi minicircle kDNA sequences integration in retrotransposon of human being LINE-1.

### DETAILED DESCRIPTION OF THE INVENTION

First, to facilitate the understanding of the present invention, some definitions narrowly related with the object of the same re supplied.

“Trypanosomatidae family protozoa”, in the context of the invention, means a member of the group consisting of Trypanosoma cruzi, Trypanosoma brucei and Leishmania sp. that parasite a host and has the capacity to integrate kDNA of its minicircles to the genome of stated host.

“reacting for extraction of DNA and analysis of the resultant fragments of cleavage with specific enzymes of restriction”, in the context of the invention, mean the used reagents in the procedures of a DNA sample extraction, in the cleavage of the DNA extracted with restriction enzymes, in the hybridization and analysis of Southern blot and others used reagents in the separation and identification of specific fragments presentd in the DNA of the sample.

“specific markers of T. cruzi kDNA integration in the host genome”, in the context of the invention, means the fragments of 1.2, 1.8 and 2.2 kb deriving of the DNA cleavage from an T. cruzi infected individual with the NsiI or EcoRI enzyme of restriction.

In a co-pendant patent, intitled “PHARMACEUTICAL COMPOSITIONS FOR the TREATMENT OF TRIPANOSSOMIASIS AND CHAGAS DISEASE”, of this depositor, are described specific compositions for the treatment of disease caused by a Trypanosomatidae family protozoa, in the chronic phase, which here is incorporated for reference. The establishment of the treatment regimen and its monitoring are based in the identification of the Trypanosomatidae family protozoa minicircles kDNA integration, what includes the T. cruzi. This identification is follow detailed.

The clinical manifestation in patients with the chronic Chagas disease takes some decades to re-echo in the health and probably it is decurrent of deleterious mutations accumulation that succeed in the genome of the individual with the Chagas disease throughout years or decades. For this reason, one efficient treatment for this disease must not only be based on the control of the parasitic load but, also, in the conditions that lead to the chronic phase of the disease and that keep it. As it will be detailed more ahead, in accordance with the present invention this objective is reached with the necessary identification of the multiple sequences of kDNA integration in the genome of the individual with the Chagas disease, making possible the choice of the adjusted treatment regimen, that, in the case of the disease in the chronic phase, is the impediment of the integration through inhibitors of metabolic pathways that hinder the mutations in the genome of the infected individual decurrent of the capacity of kDNA integration of protozoa minicircles in the host genome, in association with substances that reduce the parasitic load in the infected individual.

Although it is not the aim of the present description to be limited to a theoretical explanation, the results of the many experiences carried through until the moment have shown that the persistence of the parasite in the host infected with the T. cruzi must be a constant source of kDNA, which can be mobilized and be integrated in some sites of the genome It is believed that the persistent infection in the course of the Chagas disease represents a generating source of kDNA of new integrations. From this residual infection (source), kDNA mutations accumulates until the moment in which occur events of integration in sensible sites of the genome, destroying or forming new genes and resulting in deleterious effect for the infected individual.

In accordance with the present invention and to better study the integration of the minicircles in the human genome and to track the subsequent changes, throughout the time, in its localization, it was used a protocol using cultivated macrophages. Also they had been used T. cruzi live cells to infect a population of immortalized macrophages, followed of the recloning and maintenance of lineage free of parasite in culture of cells.

The DNA of cultures of macrophages U937 infected with T. cruzi was submitted to Southern hybridization with a probe of the conserved region of homologous minicircle (kCR). The DNA of the T. cruzi infected cells showed a distinct standard of bands that was not presented by the DNA of T. cruzi tested with the probe kCR indicating, therefore, that the DNA of the minicircle is in a different configuration in the DNA sample of infected macrophage in comparison with the kinetoplastic DNA. The samples of T. cruzi infected macrophages harvested in the fourth day after the infection had shown different standards of bands; that is, beyond the 360-pb band representing kinetoplast, bands of bigger sizes (1.2, 1.8 and 2.2 kb) had been formed with the DNA of infected macrophages, that weren't present in the DNA of the parasite.

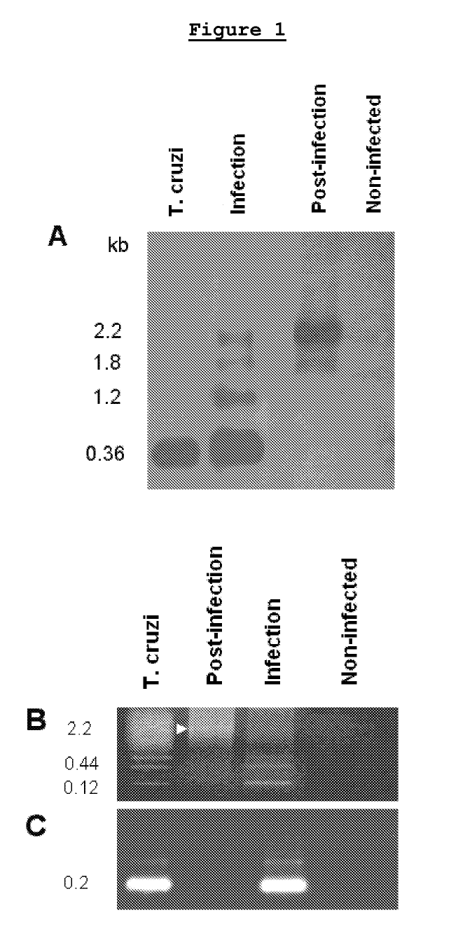

As shown in FIG. 1, the integration of the minicircles of T. cruzi can be visualized in the genome of macrophages in consequence of the infection. In (A) the Southern hybridization of macrophages NsiI digests with seven days of infection and of macrophages with thirty days (after-infection) with kCR probe in blots of agarose gel electrophoresis 0.8%. In (B), the minicircles amplification products by PCR stained with ethidium bromide can be seen, using primers S34/67. The profiles formed with the T. cruzi and the macrophage with seven days of infection are modified in the macrophages after-infection because of the absence of the 0.12 kb band and its “catâmeros”. In (C), the nDNA amplification products for PCR stained with ethidium bromide using Tcz1/2 are shown. The absence of the band of 0.36 kb in the Southern blot and of the 0.2 kb PCR products in the macrophage DNA in the thirtieth day after-infection indicates that the infection for T. cruzi had been eradicated, leaving the minicircles integrated in the genome of the hostess cell.

The determination of these characteristic bands of the T. cruzi kDNA integration in the host genome brings a unique mark in the search for a safe and efficient treatment of the Chagas disease in the chronic phase. The importance of this result after years of research became necessary the accomplishment of a series of validations as follow described.

To confirm the integration efetivation, the insertion of kDNA was made in LINE-1 elements, and the hybridization in situ was carried through by the use of L1 and kCR probe and plates of metaphase macrophages they had been infected with T. cruzi.

kDNA was co-located in LINE elements of two separate chromosomes, as well as both the probes hibridized with the same region. The control experiments had shown that nor kCR nor the L1 probes hibridized, respectively, with human nuclear DNA or kDNA of T. cruzi in non-infected metaphase macrophage chromosomes plates. Therefore, the integration event was kept inside of this lineage of cells.

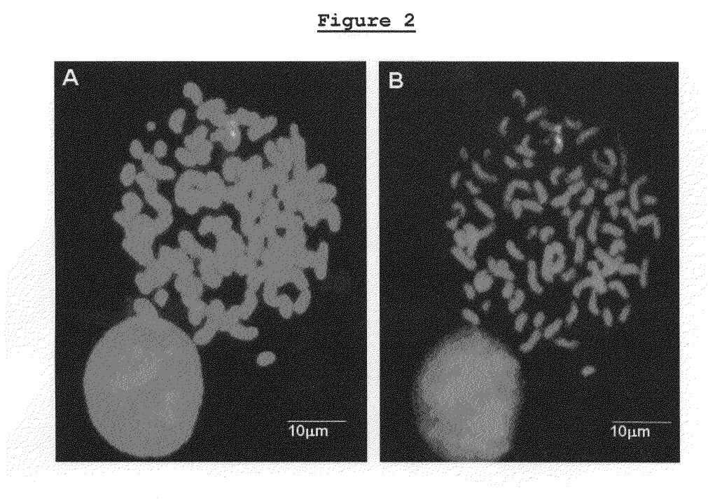

FIG. 2 shows the co-localization of the T. cruzi kDNA minicircles sequences into the macrophage chromosomes after-infection in metaphase plate. In (A) the LINE-1 showing the sprouting of fluorescent lights in two investigated chromosomes of metaphase with the specific probe L1 can be visualized (the detailed description is in the examples). In (B), is seen the kDNA sequences co-localization of minicircle inside LINE-1 with the kCR specific kDNA probe.

It was also made the verification of the events of initial integration by means of amplification for PCR using only a specific primer, the S36, with regard to a standard in the conserved region of the minicircle. The amplification products had been cloned in vector PCRII and submitted to screening with probe of T. cruzi kDNA, what resulted in the sequence of five representative clones (A to E), varying from 527 to 700 pb. These samples presented the segment ORF2 of LINE-1 between the most external regions of kDNA. Each clone contained sequences of minicircles DNA and LINE-1 (GenBank numbers AF002199 the AF002203) loaded with SINEs (HERV, MIRs and Alu-like), suggesting that the minicircle insertions had occurred inside of these highly repetitive elements.

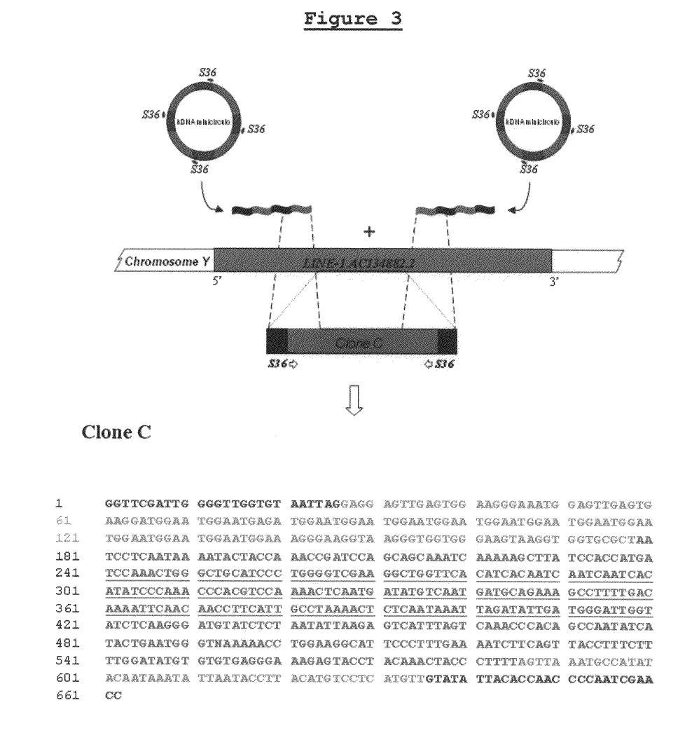

FIG. 3 shows to the schematical representation of the T. cruzi minicircles kDNA sequences integration in LINE-1 human being retrotransposon. The truncated fragments of minicircles kDNA found in the copy of LINE-1 in chromosome Y appear in clone C that shows the primer of the conserved region S36 (dark blue) in both the extremities followed by the variable regions of the minicircle (light blue) and by LINE-1 sequence (green).

This important comprehension was the base for the establishment of the method and kit of diagnosis for the identification of the T. cruzi minicircles kDNA integration and for the monitoring of the chronic treatment of tripanossomiasis according to the present invention.

The method of diagnosis and monitoring of the chronic tripanossomiasis treatment, including the chronic Chagas disease, comprises the following stages: (i) to determine the specific markers of the kDNA integration of the Trypanosomatidae family protozoa into the host genome; (ii) to extract the genomic DNA of the individual sample infected by the Trypanosomatidae family parasite; (iii) to cleave the genomic DNA with enzymes of specific restrictions and to separate the fragments from the resulting mixture; (iv) to submit the fragments under purification and under the analysis of Southern blot; (v) to hybridize the fragments with a complementary radiomarked probe (kCR) and (vi) to carry through the comparative analysis of the fragments profile of the hybridized genomic DNA with the specific markers of the kDNA integration of the Trypanosomatidae family parasite. The Trypanosomatidae family parasite is selecionated from the group of T. cruzi, T. brucei and Leishmania sp.

As previously mentioned, in the case of the T. cruzi parasite, the specific markers of kDNA integration of T. cruzi in the host are the fragments of the 2.2, 1.8 and 1.2 kb minicircles deriving from the DNA of the macrophage infected with T. cruzi and cleaved with NsiI or EcoRI.

The strategy of treatment monitoring of chronic tripanossomiasis, as defined in the above described method, also allows the distinguishing diagnosis among the diverse infections caused by Trypanosomatidae. It makes possible to generate a new version of the PCR technology with variation of primers originated of kDNA sequences and LINE-1 sequences. These curly sequences can be deduced from the integrations of chimeras in the regions of justaposition of the kDNA mutation in the host genome. This method has as advantages in relation to the known methods of the state of the technique: a) the alternation of curly sequences (primers) for amplification of the mutation in the infected individual genome, for example, in the case of T. cruzi, of the Chagas disease; b) especificity of the curly sequence of kDNA that only identifies the complementary DNA of the parasite, for example, of the Trypanosoma cruzi.

The monitoring of the tripanossomiasis chronic treatment is carried through by means of a diagnosis kit. The kit of the present invention includes all the reagents necessary to allow the identification of the kDNA integration of the parasite in the host genome for the described method. The kit consists of specific markers for the detention of the stated integration and, additionally, are supplied reacting and additives normally used in the PCR technique, as for example, appropriate nucleotides, such as dGTP (desoxiguanidin-triphosphate), dATP (desoxiadenosin-triphosphate), dCTP (desoxicitidin-triphosphate), and dTTP (desoxitimidin-triphosphate); appropriate solutions (for example, 10 mM of Tris-HCl, 50 mM of KCl, 1.5 mM of MgCl2, pH 8.5); Taq DNA polymerase; endonucleasis to digest the products of the PCR; and in the Southern blot technique, for example, marked probe and reacting for the accomplishment of blot. It will be still supplied a manual of instructions with a protocol to be used in the test, with a illustrative figure of the waited results.

As previously mentioned, in the case of American tripanossomiasis the etiologic agent of the Chagas disease is the T. cruzi protozoa whose markers of kDNA integration of the parasite in the host genome are the 1.2, 1.8 and of 2.2 kb fragments gotten by the cleavage of the gennomic DNA of human macrophages infected by T. cruzi with the enzyme of specific restriction NsiI or EcoRI. As detailed above, after a period of the intracellular infection, the inoculated macrophages with tripomastigotos of the T. cruzi had been harvested for extraction of the genomic DNA, which was cleaved with the NsiI enzyme having produced the 1.2, 1.8 and of 2.2 kb fragments that are specific of the kDNA integration of T. cruzi in the human genome.

The obtation techniques of the genomic DNA, its digestion and hybridization of the fragments are widely known of the technician in molecular biology and can be found in reference work as such as in Sambrook et al. (1989). In the particular cases, for example, of the DNA and kDNA extraction of Trypanosoma cruzi and Leishmania, the references are supplied in the description of the procedure. The detailing of the general and particular techniques of the organisms is provided in the supplied examples.

It is important to mention that the amplifications of sequences of nuclear kDNA and DNA of T. cruzi had been carried through using specific primers, being that the pairs of primers S34/67 and S35/36 (Sturm, N. R., Degrave, W., Morel, C., e Simpson, L. (1989). Sensitive detection and schizodeme classification of Trypanosoma cruzi cells by amplification of kinetoplast minicircle DNA sequences: use in diagnosis of Chagas disease. Mol. Biochem. Parasitol., 33: 205-214) amplify kDNA and the pairs of primers TcZ1/2 (Moser D R, Kirchhoff L V, Donelson J E. 1989. Detection of Trypanosoma cruzi by DNA amplification using the polymerase chain reaction. J. Clin. Microbiol. 27: 1477-82) amplify a highly repetitive sequence of nuclear DNA of the parasite.

The sequences of primers used in the amplification of kDNA of T. cruzi were:

| | |
|---|---|
| S34: | 5′ ACA CCA ACC CCA ATC GAA CC 3′ |
| S35: | 5′ ATA ATG TAC GGG (T/G)GA GAT GC 3′ |
| S36: | 5′ GGT TCG ATT GGG GTT GGT G 3′ |
| S67: | 5′ GGT TTT GGG AGG GG(G/C) (G/C) (T/G)T C 3′. |

The sequences of primers used in the amplification of the nuclear DNA of T. cruzi were:

| | |
|---|---|
| TcZ1: | 5′ GAG CTC TTG CCC CAC ACG GGT GCT 3′ |
| TcZ2: | 5′ CCT CCA AGC AGC GGA TAG TTC ACG 3′ |

The amplification conditions must be standardized for the obtantion of the correct sequences. In the examples the protocol of these amplifications is detailed.

The treatment of the sample of the individual infected by T. cruzi is made using the same procedure applied to the human macrophages for the obtainment of the specific markers of the kDNA integration of T. cruzi.

As previously mentioned, the discovery that, in the chronic phase of the Chagas disease,occurs the kDNA integration of the parasitic T. cruzi in the host genome came to clarify the difference in the reaction of patients infected by the parasite to the treatment with antiparasitic drugs, evidencing the differentiation of cure of the acute and chronic phases.

Therefore, the present invention comes to revolutionize the treatment of the Chagas disease in a way that it makes the cure by the interruption of the kDNA integration in the genome of the infected individual associated with the parasitary load reduction. In other words, the present invention makes possible the treatment of the disease of the before unavailable chronic Chagas.

To understand the premises that had led to the present invention, it follows a brief description of the use of the host mechanisms for the parasitic T. cruzi since the infection.

Experimental evidences show that the integration of T. cruzy kDNA minicircles sequences in the genome of the hostess cell is a phenomenon dependent of energy. The metabolic stress represented by the penetration of the protozoa in the cytoplasm of the hostess cell generates biochemist modifications in the cell. The molecular interactions, possibly between glycoproteins presented in the membrane of the protozoa and a present specific ligant in the surface of the hostess cell activate the metabolic pathways associated with the increase of the oxygen consumption and glucose. The energy production, initiated in the Krebs cycle, is used in all the compartments of the cell that answers to the stimulaton of the intracellular infection. In fact, the activation of multiple pathways of signals translation was recognized, having been verified that phosphatasis and phosphorilasis (protein-quinases) have a basic role in the horizontal transference of kDNA. However, the quinases are what regulate the differentiation and cellular division and, consequently, have an active role in this integration. This activity was proven by means of the specific inhibitor work of metabolic pathways that had hindered the transference of the DNA of the T. cruzi for the hostess cell.

The present invention is additionally illustrated by the followed examples that are mere a way of exemplifying the invention, without the intention to limit its target. Certain modifications and equivalents will be evidentes for those specialists in the technique and must be considered as enclosed within the target of the present invention.

### Examples

### Example 1

Infection of Macrophages with T. cruzi

The trypomastigotas forms of T. cruzi had been used to infect the U937 macrophages in the 5:1 ratio that resulted in the infection and survival of the phagocitic cells (as described in Teixeira et al., 1994). After four weeks the macrophages did not present the free parasite in the sobrenadant on the culture means, what was also confirmed by inoculation in mice. The DNA was extracted of the infected macrophages and the eradication of the infection was confirmed by PCR and Southern blot with primers and specific probe. Equally DNA of free macrophages of infection for use was extracted as control.

In the DNA control experiments, the promastigotes forms of L. braziliensis braziliensis (LTB 300) had been used to infect the macrophages in the 5:1 ratio. The cells had been kept in 37° C. in DMEM, pH 7.2 and SBF 10%. For the obtainment of the DNA of promastigotes, the culture was also made in the same concentration and in 24° C. The DNA of the macrophages free of infection was used as control.

### Example 2

Extraction of the Genomic DNA of the Macrophages

The macrophages had been processed in accordance with the described method for Sambrook et al. (1989). Two washings with TBS had been made (Tris-HCL 20 mM pH 7.2, NaCl 0.5M) the 3500×g per 15 minutes and the sediment was hanged again in 2 ml of extraction drain plug (Tris-HCl 1 mM pH 8.0, EDTA 0.1M pH 8.0, SDS a 0.5%, RNAse 200 μg/ml). After 1 hour at 37° C. incubation, it was added K proteinase in a final concentration of 100 μg/ml, continuing the incubation for more 12 hours. The cells had been submitted to two extractions with equal volume of chlorophane (phenol:chloroform:isoamilic alcohol; ratio 25:24:1), to the ambient temperature and under light agitation. The organic and watery phases had been separated for the 5000 centrifugalization×g per 10 minutes. The DNA was precipitated with 1/10 v of sodium acetate 3M pH 4.7 and 2.5v of 100% ice ethanol, and after 30 minutes of 80° C. incubation, it was sedimented for the 12000 centrifugalization×g 4° C. per 15 minutes.

The sediment was washed twice with 70% ice ethanol, dry and later hanged again in drain plug TE (10 Tris-HCl mM pH 8.0, EDTA 1 mM pH 8.0). The DNA was analyzed by eletroforesis in agarose gel 1% and storaged at 4° C.

### Example 3

Extraction of the DNA of T. cruzi and Leishmania

The epimastigotes forms of T. cruzi and promastigotes of L. braziliensis (control) grown in medium LIT and DMEM, respectively, had been dried in 1500×g per 15 minutes and the sediment washed twice with TBS and hanged again in drain plug of lyse in the concentration of 5×107 cells/ml of solution. It was made an incubation at 37° C. for 1 hour and, later, they had added to 100 μg/ml of K proteinasis and was incubated for more for 12 hours. It was proceeded two extractions from chlorophane, followed of an extraction of chlorophill. The DNA was precipitated with sodium acetate 3M pH 4.7 and of 100% ice ethanol and the sediment was washed twice with ice ethanol 70%. After dried, the DNA was hanged again in drain plug TE, analyzed for eletroforesis in agarose gel 1% and storaged at 4° C.

### Example 4

Extration of kDNA of T. cruzi and Leishmania

kDNA of T. cruzi and L. braziliensis was extracted according to methodology described by Perez-Morga & Englund, P. T. (1993) (Perez-Morga, D. L and Englund, P. T. (1993). The attachment of minicircles to kinetoplast DNA networks during replication. Cell 74:703-711). An amount of 5×10⁷ epimastigotes forms and promastigotes was dried at 1500×g per 15 minutes and the sediment was washed twice with PBS. After that, the sediment was hanged again in 630 μl of drain plug NET100 (Tris-HCl 10 mM pH 8.0, 100 EDTA mM pH 8.0, NaCl 100 mM) and the cells had been lysed with 71 μl of SDS 10%, having been added 7 μl of the K proteinasis 20 mg/ml.

The lysed solution was incubated at 37° C. during 12 hours and, after incubation, delicately was homogeneized by approximately 10 times with the aid of a syringe. After that it was added 690 μl of NET100+20% of sacarosis. The mixture was dried at 14000 rpm per 15 minutes. After that, the sobrenadant was removed carefully, leaving approximately 30 μl. Finally, 690 μl of NET100 had been added again+20% of sacarosis, repeating the centrifugalization.

After the centrifugalization, the sediment was hanged again in 1000 μl of distilled water, following two extractions of chlorophane and chlorophill. The kDNA was precipitated with 2.5v of ethanol at 100% and 0.1 v of sodium acetate 3M pH 8.0. The sediment was washed twice with ethanol 70% and hanged again in 200 μl of drain plug TE. The kDNA extracted was kept at 4° C.

### Example 5

Quantification, Enzymatic Digestion and Eletrophoretic Analysis of the DNA

The samples of genomic DNA had been quantified and analyzed related to its quality and integrity through eletrophoresis in agarose gel 0.8%, in drain plug TAE (90 Tris-acetate mM pH 8.0, EDTA 25 mM). Moreover, the quantification of the DNA also was made through spectrophotometric method, in accordance with Sambrook et al. 1989.

For the enzymatic digestion, restriction enzymes had been used (acquired from Invitrogen), following the orientations of the manufacturer, that is, for each μg of DNA it was used from 2 to 3 units of enzyme. The digestion products had been incubateds from 4 to 12 hours and later separate by eletrophoresis in agarose gel.

The fragments of DNA had been excised of the agarose gel after the eletrophoretic separation and pured using kit “DNA Purification System” (Promega) and proceeding in accordance with the instructions from the manufacturer.

### Example 6

DNA Analysis by Southern Blot

After eletrophoretic separation, the DNA was transferred to a membrane of nylon Biodyne B (Invitrogen) using the technique of alkaline transference following the teachings of Sambrook et al., 1989. Summarily, the technique consists of using a denaturing solution (NaOH 0.4 M) that, for capillarity, transfers the DNA of the agarose gel to the membrane. In the case of the genomic DNA, the previous depurination of the DNA with a solution of HCl 0.25M is necessary. After the transference, the DNA was fixed by the drying of the membrane that was kept to the ambient temperature until its use.

### Example 7

Obtainment of the Used Probe in the Hybridization

The probes had been marked through the kit “Random Primer Labelling System” (Invitrogen) where there is the insertion of [α³²P] dATP in the sequence of the DNA mold ribbon synthecized through the activity of polimerasis of the Klenow enzyme and the random hexamerics primers presence. The reaction was carried through in accordance with the instructions of the manufacturer of the kit, as followed summarized:

30 ng of DNA, in a final volume of 25 μl, had been denaturated at 100° C. per 10 minutes and later placed in ice;

It was added 2 μl of dCTP, 2 μl of dGTP and 2 μl of dTTP; 15 μl of drain plug; 3 μl of [α³²P] dATP and 1 μl of Klenow;

the reaction was incubated for 3 hours at 25° C. and stopped for the addition of 5 μl of drain plug and the resultant products (probes) were kept at −20° C.

The probes had been purified by chromatography in Sephadex G-50 column (Sambrook et al., 1989) and the radioactive incorporation was confirmed by cintilografy. The probes had been used under the limits of concentration from 1 to 2×106 cpm/ml of hybridization solution, and the gotten specific activities had been always above of 10⁸ cpm/μg of DNA.

### Example 8

Hybridization

The membranes were pre-hibridized during 4 hours at 65° C. in solution of SDS 0.5%, Solution of Denhart 5× and 100 μg/ml of salmon DNA according to the manufacturer instructions of the Biodyne B membrane (Invitrogen).

The denaturized probes were to the solution and the hybridization continued for 18 hours at 65° C. The washings of the membranes had been carried through with increasing degrees of astringency to remove probes that weren't connected to the membrane.

It was made two washings with SSC2'/SDS at 0.1% at 65° C. per 15 minutes, followed of two washings with SSC0.1×/SDS at 0.1% at 65° C. per 15 minutes. The humid membranes had been involved in plastic film of PVC and displayed in metallic cassette with film (Kodak T-MAT) sensible to X rays 80° C. The exposition varied from 12 hours to 7 days and, after that, the film was disclosed. In some cases the membranes had been DES-hibridized with solution of phormamide 50% (v/v), SSC2×, SDS 1% (p/v) 68° C. for 12 hours, following a a fast laudering with SSC0.1×/SDS at 0.1% to remove the phormamide. The membrane was kept at 4° C. until the moment of its use.

### Example 9

PCR and Amplification of Sequences of kDNA and Nuclear DNA of T. cruzi

The amplifications of sequences of kDNA and nuclear DNA of T. cruzi had been carried through using specific primers previously mentioned, that is the sequences of primers S34 (5′ ACA CCA ACC CCA ATC GAA CC 3′); S35 (5′ ATA ATG TAC GGG (T/G)GA GAT GC 3′); S36 (5′ GGT TCG ATT GGG GTT GGT G 3′) and S67 (5′ GGT TTT GGG AGG GG(G/C) (G/C)(T/G)T C 3′) for the amplification of the kDNA ofe T. cruzi (Sturm et al., 1989) and TcZ1 (5′ GAG CTC TTG CCC CAC ACG GGT GCT 3′) e TcZ2 (5′ CCT CCA AGC AGC GGA TAG TTC ACG 3′) for the amplification of the nuclear DNA of T. cruzi (Moser et al., 1989).

The amplifications had been standardized under the following conditions: 100 ng of macrophage DNA (template) had been used and the reagents of the PCR kit of the Invitrogen; drain plug of reaction (KCl 50 mM, 9.0 Tris-HCl 10 mM pH and MgCl2 1.5 mM), 100 ng of each primer, dNTPs 0.2 mM and 2.5 units of Taq DNA polymerasis. It was included in all the reactions the controls, negative and positive, in which were used the quantiiy of 100 pg of total DNA of T. cruzi Berenice. All the PCR reactions were performed in the thermocicler model PTC-100 of MJ Research following this program:

94° C./5 minutes

32 cicles (94° C./30 seconds, Tm° C. of primer/1 minute, 72/1 minute)

72° C./7 minutes

4° C.

The PCR reactions had been made in third copy and the amplified products had been visualized after eletrophoresis in agarose gel stained at 1% with 0.5 mg/ml of ethidium bromide. After that, these amplified products had been transferred to a hibridized nylon membrane and with specific probes.

1. A method of diagnosis and monitoring of the treatment of Chagas disease in the chronic phase, said method comprising the use of specific genomic markers for the determination of the occurrence of the kDNA integration of the T. cruzi in the genome of the host.

2. The method of diagnosis and monitoring according to claim 1, wherein said method comprises the following stages:
(i) to determine the specific markers of the kDNA integration of the Trypanosomatidae family protozoa into the genome of the host;
(ii) to extract the genomic DNA of the T. cruzi infected individual sample;
(iii) to cleave the genomic DNA with enzymes of specific restrictions and to separate the mixture of resultant fragments;
(iv) to submit the fragments under purification and under the analysis of Southern blot;
(v) to hybridize the fragments with a complementary probe (kCR) radiomarked; and
(vi) to carry through the comparative analysis of the fragments profile of the hybridized genomic DNA with the specific markers of the kDNA integration of the Trypanosomatidae family parasite.

3. The method of diagnosis and monitoring according to claim 2, wherein the specific genomic markers are the 1.2, 1.8 and 2.2 kb fragments gotten by the genomic DNA cleavage of human macrophages infected by T. cruzi with the NsiI or EcoRI specific restriction enzyme.

4. A method of diagnosis and monitoring of the disease treatment caused by the Trypanosomatidae family protozoa in the chronic phase, said method comprising the use of specific genomic markers for the determination of the kDNA integration occurrence of the stated protozoa in the genome of the host.

5. The method of diagnosis and monitoring according to claim 4, wherein said method comprises the following stages:
(i) to determine the specific markers of the kDNA integration of the Trypanosomatidae family protozoa into the genome of the host;
(ii) to extract the genomic DNA of the Trypanosomatidae family parasite infected individual sample;
(iii) to cleave the genomic DNA with enzymes of specific restrictions and to separate the mixture of resultant fragments;
(iv) to submit the fragments under purification and under the analysis of Southern blot;
(v) to hybridize the fragments with a complementary probe (kCR) radiomarked; and
(vi) to carry through the comparative analysis of the fragments profile of the hybridized genomic DNA with the specific markers of the kDNA integration of the Trypanosomatidae family parasite in the genome of the host.

6. A kit for diagnosis and monitoring of the treatment of chronic Chagas disease, said kit comprising:
(i) specific genomic markers of the kDNA integration of the T. cruzi in the genome of the host;
(ii) reagents and additive for the DNA extraction and analysis of the cleavage resulting fragments with specific restriction enzymes; and
(iii) instructions for the fragments extraction and analysis.

7. The kit according to claim 6, wherein the specific genomic markers are the 1.2, 1.8 and 2.2 kb fragments gotten by the cleavage of the genomic DNA of human macrophages infected by T. cruzi with the NsiI or EcoRI specific restriction enzyme.

8. The kit according to claim 6, wherein the reagents and additives are used in the PCR and Southern blot techniques.

9. A kit for diagnosis and monitoring of the treatment of a disease caused by one of the Trypanosomatidae family members, said kit comprising:
(i) specific genomic markers of the kDNA of the referred family member in the genome of the host;
(ii) reagents and additive for the DNA extraction and analysis of the cleavage resulting fragments with specific restriction enzymes; and
(iii) instructions for the fragments extraction and analysis.


SEQUENCE LISTING (7 sequences)

SEQ ID NO: 1 - DNA, 20 bp, Artificial Sequence
  Synthetic oligonucleotide primer
acaccaaccccaatcgaacc

SEQ ID NO: 2 - DNA, 20 bp, Artificial Sequence
  Synthetic oligonucleotide primer
ataatgtacgggkgagatgc

SEQ ID NO: 3 - DNA, 19 bp, Artificial Sequence
  Synthetic oligonucleotide primer
ggttcgattggggttggtg

SEQ ID NO: 4 - DNA, 19 bp, Artificial Sequence
  Synthetic oligonucleotide primer
ggttttgggaggggssktc

SEQ ID NO: 5 - DNA, 24 bp, Artificial Sequence
  Synthetic oligonucleotide primer
gagctcttgccccacacgggtgct

SEQ ID NO: 6 - DNA, 24 bp, Artificial Sequence
  Synthetic oligonucleotide primer
cctccaagcagcggatagttcacg

SEQ ID NO: 7 - DNA, 662 bp, Artificial Sequence
  Synthetic polynucleotide
ggttcgattggggttggtgtaattaggaggagttgagtggaagggaaatggagttgagtgaaggatggaatggaatgagatggaatggaatggaatggaatggaatggaatggaatggaatggaatggaatggaatggaaagggaaggtaagggtggtgggaagtaaggtggtgcgctaatcctcaataaaatactaccaaaccgatccagcagcaaatcaaaaagcttatccaccatgatccaaactgggctgcatccctggggtcgaaggctggttcacatcacaatcaatcaatcacatatcccaaacccacgtccaaaactcaatgatatgtcaatgatgcagaaagccttttgacaaaattcaacaaccttcattgcctaaaactctcaataaattagatattgatgggatttgtatctcaagggatgtatctctaatattaagagtcatttagtcaaacccacagccaatatcatactgaatgggtaaaaaacctggaaggcattccctttgaaaatcttcagttacctttcttttggatatgtgtgtgagggaaagagtacctacaaactacccttttagttaaatgccatatacaataaatattaataccttacatgtcctcatgttgtatattacaccaaccccaatcgaacc